(19) United States
(12) Patent Application Publication
(10) Pub. No.: US 2023/0009425 A1
(43) Pub. Date: Jan. 12, 2023

(54) A PRODUCING DEVICE AND PREPARING PROCESS OF A BLACK PLASTER

(71) Applicant: Shandong Zhushi Pharmaceutical Group Co., Ltd., Heze (CN)

(72) Inventors: Kunfu ZHU, Heze (CN); Lei ZHU, Heze (CN); Junhui ZHU, Heze (CN); Meng ZHU, Heze (CN); Fei XUE, Heze (CN)

(73) Assignee: Shandong Zhushi Pharmaceutical Group Co., Ltd., Heze (CN)

(21) Appl. No.: 17/042,284

(22) PCT Filed: Jul. 24, 2020
(86) PCT No.: PCT/CN2020/104091

§ 371 Date: Sep. 28, 2020

(30) Foreign Application Priority Data
May 27, 2020 (CN) 202010459232.9

(51) Int. Cl. A61J 3/04; A61K 36/282; A61K 36/714; A61K 36/48; A61K 35/64; A61K 35/646; A61K 35/648; A61K 36/185; A61K 36/47; A61K 36/482; A61K 36/24; A61K 31/353; A61K 36/54; A61K 36/237; A61K 36/23; A61K 36/268; A61K 36/15; A61K 36/11; A61K 36/21; A61K 36/126; A61K 36/45; A61K 36/70; A61K 36/515; A61K 36/8945; A61K 36/234; A61K 36/296; A61K 36/232; A61K 36/18; A61K 36/59; A61K 36/71; A61K 36/324; A61K 36/328; A61K 36/67; A61K 31/045; A61K 33/34; A61K 9/06

(52) U.S. Cl. CPC A61J 3/04; A61K 36/282; A61K 36/714; A61K 36/48; A61K 35/64; A61K 35/646; A61K 35/648; A61K 36/185; A61K 36/47; A61K 36/482; A61K 36/24; A61K 31/353; A61K 36/54; A61K 36/237; A61K 36/23; A61K 36/268; A61K 36/15; A61K 36/11; A61K 36/21; A61K 36/126; A61K 36/45; A61K 36/70; A61K 36/515; A61K 36/8945; A61K 36/234; A61K 36/296; A61K 36/232; A61K 36/18; A61K 36/59; A61K 36/71; A61K 36/324; A61K 36/328; A61K 36/67; A61K 31/045; A61K 33/34; A61K 9/06; A61K 2236/17; A61K 2236/15; A61J 2200/42

(57) ABSTRACT

The present invention provides a black plaster producing device that comprises a workbench, longitudinal supports, a transverse bottom plate, an electric-heated thermostatic drying oven, and a storage rack structure for classified storage, a storage bucket, a stove, a boiling furnace, a furnace cover, a stirring box, a box cover, a stirring motor, a stirring pipe, a crushing blade, an adjustable partition frame structure, a rolling frame structure, a gas inlet pipe, a pipe valve, a stirring switch, a rolling switch and a receiving bucket, wherein the longitudinal supports are welded at four corners at the lower part of the workbench respectively, and the transverse bottom plate is screwed at the lower part of the longitudinal supports on the inner side. The transverse partition plate of the present invention is screwed at the central position inside the storage box, which facilitates the convenient storage of materials in the storage box during use and can classify the stored materials at the same time. The semiconductor refrigerating chip is screwed on the rear side of the inner wall at the lower part of the storage box, which facilitates the refrigeration and preservation of the materials stored at the bottom of the storage box on the inner side during use.

### FIELD OF THE INVENTION

The present invention belongs to the technical field of traditional Chinese medicine preparations, and in particularly relates to a producing device and preparing process of a black plaster.

### BACKGROUND ART

Black plaster is one of the traditional dosage forms of traditional Chinese medicine therapy in the motherland. It is listed as the first of the five major dosage forms of traditional Chinese medicine. It is a kind of external treatment of traditional Chinese medicine. Treating disease by plaster, with a history of thousands of years in China, has a unique curative effect on clinic and is still used today. Its medicinal properties directly enter into striae from pores, which does not stimulate intestines and stomach and avoid damage to liver and kidney, and have the advantages such as convenient to use, long-lasting efficacy, accurate curative effect, wide indications, and so on. It has been widely applied in individual departments such as internal medicine, surgical, gynecology and pediatrics departments, and achieved gratifying achievements.

However, the existing technology has the problems that it is inconvenient to store finished products, inconvenient to cool the materials, inconvenient to shield the materials, and inconvenient to crush the materials.

Therefore, it is very necessary to invent a producing device and preparing process of a black plaster.

### SUMMARY OF THE INVENTION

In order to solve the above-mentioned technical problems, the present invention provides a producing device and preparing process of a black plaster. to solve the problems in the existing technology that it is inconvenient to store finished products, inconvenient to cool the materials, inconvenient to shield the materials, and inconvenient to crush the materials. A black plaster producing device comprises a workbench, longitudinal supports, a transverse bottom plate, an electric-heated thermostatic drying oven, and a storage rack structure for classified storage, a storage bucket, a stove, a boiling furnace, a furnace cover, a stirring box, a box cover, a stirring motor, a stirring pipe, a crushing blade, an adjustable partition frame structure, a rolling frame structure, a gas inlet pipe, a pipe valve, a stirring switch, a rolling switch and a receiving bucket. The longitudinal supports are welded at four corners at the lower part of the workbench respectively. The transverse bottom plate is screwed at the lower part of the longitudinal supports on the inner side. The electric-heated thermostatic drying oven is screwed on the right side of the upper part of the transverse bottom plate. The storage rack structure for classified storage is installed on the left side of the upper part of the transverse bottom plate of the workbench. The storage bucket is provided under the adjustable partition frame structure and arranged at the upper part of the transverse bottom plate at the same time. The stove is screwed on the left side of the upper part of the workbench. The boiling furnace is arranged on the upper side of the stove. The furnace cover is arranged on the upper side of the boiling furnace. The stirring box is embedded in the right side of the workbench. The box cover is arranged on the upper side of the stirring box. The stirring motor is screwed at the middle-upper part of the box cover. The stirring pipe is arranged on the inner side of the stirring box and threadedly connected to the output shaft of the stirring motor at the same time. The crushing blade is welded on the left and right sides of the lower part of the stirring pipe. The adjustable partition frame structure is installed at the lower part of the stirring box. The rolling frame structure is installed at the central position of the workbench. The gas inlet pipe is plugged into the left side of the workbench and connected to the stove at the same time. The pipe valve is threadedly connected to the left side of the gas inlet pipe. The stirring switch and the rolling switch are respectively screwed on the right side of the front part of the workbench. The receiving bucket is provided under the adjustable partition frame structure and arranged on the upper side of the electric-heated thermostatic drying oven at the same time. The storage rack structure for classified storage comprises a storage box, a transverse partition plate, protective doors, a handle, a rectangular magnet piece, metal plates and a semiconductor refrigerating chip. The storage box is screwed on the left side of the upper part of the transverse bottom plate. The protective doors are respectively hinged at the upper and lower parts on the left side of the front end of the storage box. The handle is screwed at the central position on the right side of the front part of the protective door. The rectangular magnet piece is embedded in the upper and lower parts on the right side of the protective door. The metal plates are respectively embedded in the upper and lower parts and the central position on the right side of the storage box.

Preferably, the rolling frame structure comprises a connecting bucket, a lead-in bucket, a crushing frame, and a lead-out bucket, a crushing gear roller and a rotating motor. The connecting bucket is embedded in the central position of the workbench; the crushing frame is welded to the middle-lower part of the connecting bucket; the lead-out bucket is screwed at the lower part of the crushing frame; the crushing gear rollers are provided on the left and right sides inside the crushing frame; and the rotating motor are screwed on the left and right sides of the front part of the crushing frame and keyed to the central position of the crushing gear roller.

Preferably, the adjustable partition frame structure comprises a transverse insert frame, a positioning frame, and a transverse insert plate, a positioning seat and a rectangular handle, wherein the transverse frame is screwed at the lower part of the stirring box; the positioning frame is screwed on the right side of the transverse insert frame; and the rectangular handle is screwed on the right side of the transverse insert plate.

Preferably, the transverse partition plate is screwed at the central position inside the storage box.

Preferably, the semiconductor refrigerating chip is screwed on the rear side of the inner wall at the lower part of the storage box.

Preferably, the transverse insert plate penetrates the positioning frame and is inserted on the inner side of the transverse insert frame.

Preferably, there are two crushing gear rollers, and protruding tooth on the crushing gear rollers mesh with each other.

Preferably, a filter screen of 100 mesh to 110 mesh is screwed to the bottom of the lead-out bucket on the inner side.

Preferably, silicone gaskets are glued around the periphery on the rear side of the protective door.

Preferably, the lead-in bucket is screwed at the middle-upper part of the crushing frame.

Preferably, the positioning seat is screwed at the left end of the transverse insert plate, and the lower end thereof is in contact with the upper part of the connecting bucket at the same time.

Preferably, the stirring motor is electrically connected to the stirring switch. The rolling motor is electrically connected to the rolling switch. The stirring motor and the rolling motor each is a Y2-1 motor.

A preparing process of a black plaster specifically comprises the following steps:

Step 1: selecting raw materials comprising 155 parts of mugwort leaves, 130 parts of Radix aconiti Preparata, 130 parts of Radix aconiti kusnezoffii Preparata, 155 parts of Bauhinia championii Benth, 155 parts of Clematis chinensis Osbeck, 155 parts of Bombyx batryticatus, 110 parts of whole scorpion, 65 parts of Centipede, 155 parts of Garden balsam stem, 155 parts of Geranium, 155 parts of Twig and leaf of Common Claoxylon, 155 parts of Cassia twig, 110 parts of Tylophora floribunda, 110 parts of Psoralen, 155 parts of Cinnamon, 130 parts of Notopterygium incisum, 130 parts of Heracleum hemsleyanum Diels, 110 parts of Parsnips, 130 parts of Sargentodoxa cuneata, 155 parts of Asarum, 130 parts of Lignum pini nodi, 155 parts of Woodwardia japonica, 155 parts of Achyranthes bidentata, 155 parts of Artemisia, 155 parts of Hematoxylin, 110 parts of Drynaria fortunei, 130 parts of Pyrola calliantha, 155 parts of Fallopia multiflora, 155 parts of Caulis spatholobi, 155 parts of Fissistigma polyanthum, 110 parts of Gentiana macrophylla, 110 parts of Discorea nipponica Makino, 155 parts of Cnidium, 155 parts of Ardisia japonica, 155 parts of Epimedium, 155 parts of Angelica dahurica, 130 parts of Sarcandra glabra, 130 parts of Menispermaceae, 155 parts of Pulsatilla, 155 parts of raw frankincense, 155 parts of raw myrrh, 110 parts of white pepper, 55 parts of borneol and 130 parts of natural copper;

Step 2: preliminary processing of the raw materials, comprising separately extracting effective ingredients of the prescription Chinese medicine by water decocting method, boiling out a concentrated thick paste from a boiling furnace and then placing it in an electric-heated thermostatic drying oven for drying at 81 to 90 degrees Celsius, grinding it into a 160-mesh concentrated fine powder, and then mixing the powder into a plaster matrix at a ratio of two to sixty;

Step 3: secondary processing of the raw materials, comprising boiling a sesame oil in the boiling furnace to the extent that dropped water will become a pearl, adding minium at 330 degrees Celsius to make the plaster matrix, removing fire toxin using a process with a combination of water immersion, alcohol and cold storage methods, taking 30 parts by weight of the matrix and putting it into the stove with slow fire to be heated and melted, keeping the temperature between 71° C. and 80° C. to avoid the production of new fire toxin, stirring evenly with one part by weight of the extracted concentrated fine powder or fine medicine, exhausting the moisture and removing from the fire;

Step 4: packaging the finished product by putting the finished product into a packaging bag for packaging, and then putting it into the storage box.

Compared to the prior art, the present invention has the following beneficial effects:

1. In the present invention, the transverse partition plate of the present invention is screwed at the central position inside the storage box, which facilitates the convenient storage of materials in the storage box during use and can classify the stored materials at the same time.

2. In the present invention, the semiconductor refrigerating chip is screwed on the rear side of the inner wall at the lower part of the storage box, which facilitates the refrigeration and preservation of the materials stored at the bottom of the storage box on the inner side during use.

3. In the present invention, the transverse insert plate penetrates the positioning frame and is inserted on the inner side of the transverse insert frame, which facilitates the convenient shielding function of the materials stirred in the stirring box during use so as to avoid unstirred materials falling into the connecting bucket.

4. In the present invention, there are two crushing gear rollers, and the protruding teeth on the crushing gear roller mesh with each other, which facilitates the convenient crushing of the falling raw materials during use.

5. In the present invention, a filter screen of 100 mesh to 110 mesh is also screwed to the bottom of the lead-out bucket on the inner side, which facilitates the convenient filtering of materials that drip through the lead-out bucket during use.

6. In the present invention, silicone gaskets are also glued around the periphery on the rear side of the protective door, which facilitates to increase a sealing effect when the protective door is in contact with the storage box during use.

7. In the present invention, the lead-in bucket is screwed at the middle-upper part of the crushing frame, which facilitates the convenient central pouring of materials into the upper part of the crushing gear roller on the inner side during use.

8. In the present invention, the positioning seat screw is screwed at the left end of the transverse insert plate, and the lower end thereof is in contact with the upper part of the connecting bucket at the same time, which facilitates to prevent the transverse insert plate from moving up and down within the transverse insert frame during use.

### BRIEF DESCRIPTION OF THE DRAWINGS

FIG. 1 is a schematic structural view of the present invention. FIG. 2 is a schematic structural view of a storage rack structure for classified storage of the present invention.

FIG. 3 is a schematic structural view of an adjustable partition frame structure of the present invention.

FIG. 4 is a schematic structural view of a rolling frame structure of the present invention.

FIG. 5 is a flowchart of the present invention.

In the drawings:

1. Workbench; 2. Longitudinal support; 3. Transverse bottom plate; 4. Electric-heated thermostatic drying oven; 5. Storage rack structure for classified storage; 51. Storage box; 52. Transverse partition plate; 53. Protective door; 54. Handle; 55. Rectangular magnet piece; 56. metal plate; 57. semiconductor refrigerating chip; 6. storage bucket; 7. furnace; 8. boiling furnace; 9. furnace cover; 10. stirring box; 11. box cover; 12. stirring motor 13. Stirring pipe; 14. Crushing blade; 15. Adjustable partition frame structure; 151. Transverse insert frame; 152. Positioning frame; 153. Transverse insert plate; 154. Positioning seat; 155. Rectangular handle; 16. Rolling Frame structure; 161. Connecting bucket; 162. Lead-in bucket; 163. Crushing frame; 164. Lead-out bucket; 165. Crushing gear roller; 166. Rotating motor; 17. Gas inlet pipe; 18. Pipe valve; 19. Stirring switch; 20. Rolling switch; 21. Receiving bucket.

### DETAILED EMBODIMENTS

The present invention will be further described below in conjunction with the drawings.

In the drawings:

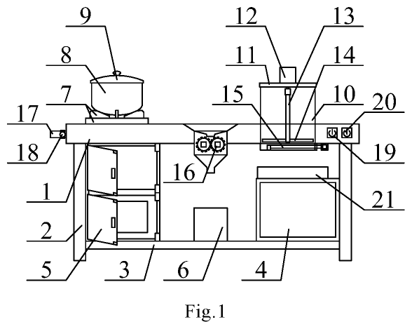

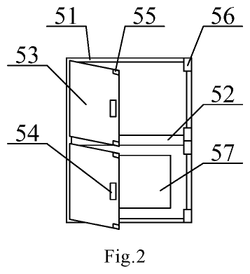

As shown in FIGS. 1 and 2, the present invention provides a black plaster producing device that comprises a workbench 1, longitudinal supports 2, a transverse bottom plate 3, an electric-heated thermostatic drying oven 4, and a storage rack structure for classified storage 5, a storage bucket 6, a stove 7, a boiling furnace 8, a furnace cover 9, a stirring box 10, a box cover 11, a stirring motor 12, a stirring pipe 13, a crushing blade 14, an adjustable partition frame structure 15, a rolling frame structure 16, a gas inlet pipe 17, a pipe valve 18, a stirring switch 19, a rolling switch 20 and a receiving bucket 21. The longitudinal supports 2 are welded at four corners at the lower part of the workbench 1 respectively. The transverse bottom plate 3 is screwed at the lower part of the longitudinal supports 2 on the inner side. The electric-heated thermostatic drying oven 4 is screwed on the right side of the upper part of the transverse bottom plate 3. The storage rack structure for classified storage 5 is installed on the left side of the upper part of the transverse bottom plate 3 of the workbench 1. The storage bucket 6 is provided under the adjustable partition frame structure 15 and arranged at the upper part of the transverse bottom plate 3 at the same time. The stove 7 is screwed on the left side of the upper part of the workbench 1. The boiling furnace 8 is arranged on the upper side of the stove 7. The furnace cover 9 is arranged on the upper side of the boiling furnace 8. The stirring box 10 is embedded in the right side of the workbench 1. The box cover 11 is arranged on the upper side of the stirring box 10. The stirring motor 12 is screwed at the middle-upper part of the box cover 11. The stirring pipe 13 is arranged on the inner side of the stirring box 10 and threadedly connected to the output shaft of the stirring motor 12 at the same time. The crushing blade 14 is welded on the left and right sides of the lower part of the stirring pipe 13. The adjustable partition frame structure 15 is installed at the lower part of the stirring box 10. The rolling frame structure 16 is installed at the central position of the workbench 1. The gas inlet pipe 17 is plugged into the left side of the workbench 1 and connected to the stove 7 at the same time. The pipe valve 18 is threadedly connected to the left side of the gas inlet pipe 17. The stirring switch 19 and the rolling switch 20 are respectively screwed on the right side of the front part of the workbench 1. The receiving bucket 21 is provided under the adjustable partition frame structure 15 and arranged on the upper side of the electric-heated thermostatic drying oven 4 at the same time. The storage rack structure for classified storage 5 comprises a storage box 51, a transverse partition plate 52, protective doors 53, a handle 54, a rectangular magnet piece 55, metal plates 56 and a semiconductor refrigerating chip 57. The storage box 51 is screwed on the left side of the upper part of the transverse bottom plate 3. The protective doors 53 are respectively hinged at the upper and lower parts on the left side of the front end of the storage box 51. The handle 54 is screwed at the central position on the right side of the front part of the protective door 53. The rectangular magnet piece 55 is embedded in the upper and lower parts on the right side of the protective door 53. The metal plates 56 are respectively embedded in the upper and lower parts and the central position on the right side of the storage box 51. The transverse partition plate 52 is screwed at the central position inside the storage box 51, which facilitates the convenient storage of materials in the storage box 51 during use and can classify the stored materials at the same time. The semiconductor refrigerating chip 57 is screwed on the rear side of the inner wall at the lower part of the storage box 51, which facilitates the refrigeration and preservation of the materials stored at the bottom of the storage box 51 on the inner side during use. Silicone gaskets are also glued around the periphery on the rear side of the protective door 53, which facilitates to increase a sealing effect when the protective door 53 is in contact with the storage box 51 during use.

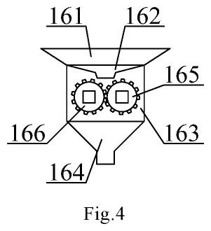

As shown in FIG. 4, in the above embodiment, specifically, the rolling frame structure 16 comprises a connecting bucket 161, a lead-in bucket 162, a crushing frame 163, and a lead-out bucket 164, a crushing gear roller 165 and a rotating motor 166. The connecting bucket 161 is embedded in the central position of the workbench 1; the crushing frame 163 is welded to the middle-lower part of the connecting bucket 161; the lead-out bucket 164 is screwed at the lower part of the crushing frame 163; the crushing gear rollers 165 are provided on the left and right sides inside the crushing frame 163; and the rotating motor 166 are screwed on the left and right sides of the front part of the crushing frame 163 and keyed to the central position of the crushing gear roller 165. There are two crushing gear rollers 165, and protruding tooth on the crushing gear rollers 165 mesh with each other, which facilitates the convenient crushing of the falling raw materials during use. A filter screen of 100 mesh to 110 mesh is also screwed to the bottom of the lead-out bucket 164 on the inner side, which facilitates the convenient filtering of materials that drip through the lead-out bucket 164 during use. The lead-in bucket 162 is screwed at the middle-upper part of the crushing frame 163, which facilitates the convenient central pouring of materials into the upper part of the crushing gear roller 165 on the inner side during use.

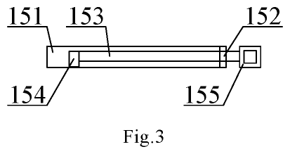

As shown in FIG. 3, in the above embodiment, specifically, the adjustable partition frame structure 15 comprises a transverse insert frame 151, a positioning frame 152, and a transverse insert plate 153, a positioning seat 154 and a rectangular handle 155. The transverse frame 151 is screwed at the lower part of the stirring box 10; the positioning frame 152 is screwed on the right side of the transverse insert frame 151; and the rectangular handle 155 is screwed on the right side of the transverse insert plate 153. The transverse insert plate 153 penetrates the positioning frame 152 and is inserted on the inner side of the transverse insert frame 151, which facilitates the convenient shielding function of the materials stirred in the stirring box 10 during use so as to avoid unstirred materials falling into the connecting bucket 161. The positioning seat 154 is screwed at the left end of the transverse insert plate 153, and the lower end thereof is in contact with the upper part of the connecting bucket 161 at the same time, which facilitates to prevent the transverse insert plate 153 from moving up and down within the transverse insert frame 151 during use.

A preparing process of a black plaster specifically comprises the following steps:

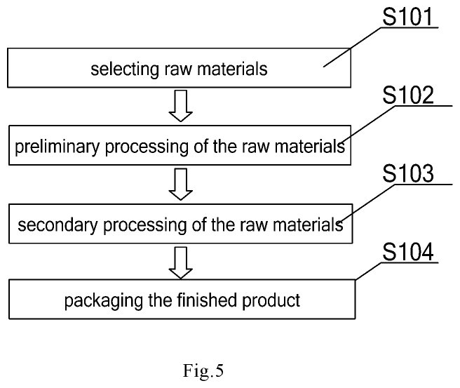

With refence to FIG. 5

S101: selecting raw materials comprising 155 parts of mugwort leaves, 130 parts of Radix aconiti Preparata, 130 parts of Radix aconiti kusnezoffii Preparata, 155 parts of Bauhinia championii Benth, 155 parts of Clematis chinensis Osbeck, 155 parts of Bombyx batryticatus, 110 parts of whole scorpion, 65 parts of Centipede, 155 parts of Garden balsam stem, 155 parts of Geranium, 155 parts of Twig and leaf of Common Claoxylon, 155 parts of Cassia twig, 110 parts of Tylophora floribunda, 110 parts of Psoralen, 155 parts of Cinnamon, 130 parts of Notopterygium incisum, 130 parts of Heracleum hemsleyanum Diels, 110 parts of Parsnips, 130 parts of Sargentodoxa cuneata, 155 parts of Asarum, 130 parts of Lignum pini nodi, 155 parts of Woodwardia japonica, 155 parts of Achyranthes bidentata, 155 parts of Artemisia, 155 parts of Hematoxylin, 110 parts of Drynaria fortunei, 130 parts of Pyrola calliantha, 155 parts of Fallopia multiflora, 155 parts of Caulis spatholobi, 155 parts of Fissistigma polyanthum, 110 parts of Gentiana macrophylla, 110 parts of Discorea nipponica Makino, 155 parts of Cnidium, 155 parts of Ardisia japonica, 155 parts of Epimedium, 155 parts of Angelica dahurica, 130 parts of Sarcandra glabra, 130 parts of Menispermaceae, 155 parts of Pulsatilla, 155 parts of raw frankincense, 155 parts of raw myrrh, 110 parts of white pepper, 55 parts of borneol and 130 parts of natural copper;

S102: preliminary processing of the raw materials, comprising separately extracting effective ingredients of the prescription Chinese medicine by water decocting method, boiling out a concentrated thick paste from a boiling furnace and then placing it in an electric-heated thermostatic drying oven for drying at 81 to 90 degrees Celsius, grinding it into a 160-mesh concentrated fine powder, and then mixing the powder into a plaster matrix at a ratio of two to sixty;

S103: secondary processing of the raw materials, comprising boiling a sesame oil in the boiling furnace to the extent that dropped water will become a pearl, adding minium at 330 degrees Celsius to make the plaster matrix, removing fire toxin using a process with a combination of water immersion, alcohol and cold storage methods, taking 30 parts by weight of the matrix and putting it into the stove with slow fire to be heated and melted, keeping the temperature between 71° C. and 80° C. to avoid the production of new fire toxin, stirring evenly with one part by weight of the extracted concentrated fine powder or fine medicine, exhausting the moisture and removing from the fire;

S104: packaging the finished product by putting the finished product into a packaging bag for packaging, and then putting it into the storage box.

Working Principle During use, the raw materials are put into the boiling furnace 8. The pipe valve 18 is opened to make the external gas to be introduced into the stove 7 via the gas inlet pipe 17. The stove 7 is fired up so as to boil the materials in the boiling furnace 8 several times. The residue is introduced into the crushing frame 163 at the lead-in bucket 162 through the connecting bucket 161, and at the same time, the rolling switch 20 is turned on and thus the rotating motor 166 is used to drive the crushing gear roller 165 to rotate so as to crush the residue, and the internal juice is filtered through a filter mesh at the bottom of the lead-out bucket 164 and drop into the storage bucket 6, and then is put into the boiling furnace 8 to be boiled into a concentrated thick paste. Finally, the concentrated thick paste is put into the electric-heated thermostatic drying oven 4 to be dried at the temperature that is controlled within 81 to 90 degrees Celsius. Finally, the stirring switch 19 supplies power to the stirring motor 12, and at the same time, the box cover 11 is opened so as to put the dried materials into the stirring box 10, and the stirring motor 12 is used to drive the crushing blade 14 through the stirring pipe 13 to rotate to crush the materials. Finally, the rectangular handle 155 is pulled to the right so as to drop the powder materials from the transverse insert frame 151 into the receiving bucket 21. The concentrated fine powder is extracted. The sesame oil is boiled in the boiling furnace 8 to the extent that dropped water will become a pearl. Minium is added at 330 degrees Celsius to make the plaster matrix. After fire toxin is removed using a process with a combination of water immersion, alcohol and cold storage methods, 30 parts by weight of the matrix is taken and put into the stove with slow fire to be heated and melted, keeping the temperature between 71° C. and 80° C. to avoid the production of new fire toxin, and is stirred with one part by weight of extracted concentrated fine powder or fine medicine, and removed from the fire after exhausting the moisture.

Similar technical solutions, that are designed using the technical solution described in the present invention, or by those skilled in the art under the inspiration of the technical solution of the present invention and can achieve the above technical effects, fall into the protection scope of the present invention.

1. A black plaster producing device, characterized in that:
the black plaster producing device comprises a workbench (1), longitudinal supports (2), a transverse bottom plate (3), an electric-heated thermostatic drying oven (4), and a storage rack structure for classified storage (5), a storage bucket (6), a stove (7), a boiling furnace (8), a furnace cover (9), a stirring box (10), a box cover (11), a stirring motor (12), a stirring pipe (13), a crushing blade (14), an adjustable partition frame structure (15), a rolling frame structure (16), a gas inlet pipe (17), a pipe valve (18), a stirring switch (19), a rolling switch (20) and a receiving bucket (21),
the longitudinal supports (2) are welded at four corners at the lower part of the workbench (1) respectively;
the transverse bottom plate (3) is screwed at the lower part of the longitudinal supports (2) on the inner side;
the electric-heated thermostatic drying oven (4) is screwed on the right side of the upper part of the transverse bottom plate (3);
the storage rack structure for classified storage (5) is installed on the left side of the upper part of the transverse bottom plate (3) of the workbench (1);
the storage bucket (6) is provided under the adjustable partition frame structure (15) and arranged at the upper part of the transverse bottom plate (3) at the same time;
the stove (7) is screwed on the left side of the upper part of the workbench (1);
the boiling furnace (8) is arranged on the upper side of the stove (7);
the furnace cover (9) is arranged on the upper side of the boiling furnace (8);
the stirring box (10) is embedded in the right side of the workbench (1);
the box cover (11) is arranged on the upper side of the stirring box (10);
the stirring motor (12) is screwed at the middle-upper part of the box cover (11);
the stirring pipe (13) is arranged on the inner side of the stirring box (10) and threadedly connected to the output shaft of the stirring motor (12) at the same time;
the crushing blade (14) is welded on the left and right sides of the lower part of the stirring pipe (13);
the adjustable partition frame structure (15) is installed at the lower part of the stirring box (10);
the rolling frame structure (16) is installed at the central position of the workbench the gas inlet pipe (17) is plugged into the left side of the workbench (1) and connected to the stove (7) at the same time;
the pipe valve (18) is threadedly connected to the left side of the gas inlet pipe (17);
the stirring switch (19) and the rolling switch (20) are respectively screwed on the right side of the front part of the workbench (1);
the receiving bucket (21) is provided under the adjustable partition frame structure (15) and arranged on the upper side of the electric-heated thermostatic drying oven (4) at the same time;
the storage rack structure for classified storage (5) comprises a storage box (51), a transverse partition plate (52), protective doors (53), a handle (54), a rectangular magnet piece (55), metal plates (56) and a semiconductor refrigerating chip (57);
the storage box (51) is screwed on the left side of the upper part of the transverse bottom plate (3);
the protective doors (53) are respectively hinged at the upper and lower parts on the left side of the front end of the storage box (51);
the handle (54) is screwed at the central position on the right side of the front part of the protective door (53);
the rectangular magnet piece (55) is embedded in the upper and lower parts on the right side of the protective door (53);
the metal plates (56) are respectively embedded in the upper and lower parts and the central position on the right side of the storage box (51).

2. The black plaster producing device according to claim 1, characterized in that: the rolling frame structure (16) comprises a connecting bucket (161), a lead-in bucket (162), a crushing frame (163), and a lead-out bucket (164), a crushing gear roller (165) and a rotating motor (166), the connecting bucket (161) is embedded in the central position of the workbench (1);
the crushing frame (163) is welded to the middle-lower part of the connecting bucket (161);
the lead-out bucket (164) is screwed at the lower part of the crushing frame (163); the crushing gear rollers (165) are provided on the left and right sides inside the crushing frame (163); and the rotating motor (166) are screwed on the left and right sides of the front part of the crushing frame (163) and keyed to the central position of the crushing gear roller (165).

3. The black plaster producing device according to claim 1, characterized in that the adjustable partition frame structure (15) comprises a transverse insert frame (151), a positioning frame (152), and a transverse insert plate (153), a positioning seat (154) and a rectangular handle (155), wherein the transverse frame (151) is screwed at the lower part of the stirring box (10); the positioning frame (152) is screwed on the right side of the transverse insert frame (151); and the rectangular handle (155) is screwed on the right side of the transverse insert plate (153).

4. The black plaster producing device according to claim 1, characterized in that the transverse partition plate (52) is screwed at the central position inside the storage box (51).

5. The black plaster producing device according to claim 1, characterized in that the semiconductor refrigerating chip (57) is screwed on the rear side of the inner wall at the lower part of the storage box (51).

6. The black plaster producing device according to claim 3, characterized in that the transverse insert plate (153) penetrates the positioning frame (152) and is inserted on the inner side of the transverse insert frame (151).

7. The black plaster producing device according to claim 2, characterized in that there are two crushing gear rollers (165), and protruding tooth on the crushing gear rollers (165) mesh with each other.

8. The black plaster producing device according to claim 2, characterized in that the lead-in bucket (162) is screwed at the middle-upper part of the crushing frame (163).

9. The black plaster producing device according to claim 3, characterized in that the positioning seat (154) is screwed at the left end of the transverse insert plate (153), and the lower end thereof is in contact with the upper part of the connecting bucket (161) at the same time.

10. A preparing process of the black plaster according to claim 1, comprising the following steps:
Step 1: selecting raw materials comprising 155 parts of mugwort leaves, 130 parts of Radix aconiti Preparata, 130 parts of Radix aconiti kusnezoffii Preparata, 155 parts of Bauhinia championii Benth, 155 parts of Clematis chinensis Osbeck, 155 parts of Bombyx batryticatus, 110 parts of whole scorpion, 65 parts of Centipede, 155 parts of Garden balsam stem, 155 parts of Geranium, 155 parts of Twig and leaf of Common Claoxylon, 155 parts of Cassia twig, 110 parts of Tylophora floribunda, 110 parts of Psoralen, 155 parts of Cinnamon, 130 parts of Notopterygium incisum, 130 parts of Heracleum hemsleyanum Diels, 110 parts of Parsnips, 130 parts of Sargentodoxa cuneata, 155 parts of Asarum, 130 parts of Lignum pini nodi, 155 parts of Woodwardia japonica, 155 parts of Achyranthes bidentata, 155 parts of Artemisia, 155 parts of Hematoxylin, 110 parts of Drynaria fortunei, 130 parts of Pyrola calliantha, 155 parts of Fallopia multiflora, 155 parts of Caulis spatholobi, 155 parts of Fissistigma polyanthum, 110 parts of Gentiana macrophylla, 110 parts of Discorea nipponica Makino, 155 parts of Cnidium, 155 parts of Ardisia japonica, 155 parts of Epimedium, 155 parts of Angelica dahurica, 130 parts of Sarcandra glabra, 130 parts of Menispermaceae, 155 parts of Pulsatilla, 155 parts of raw frankincense, 155 parts of raw myrrh, 110 parts of white pepper, 55 parts of borneol and 130 parts of natural copper;
Step 2: preliminary processing of the raw materials, comprising separately extracting effective ingredients of the prescription Chinese medicine by water decocting method, boiling out a concentrated thick paste from a boiling furnace and then placing it in an electric-heated thermostatic drying oven for drying at 81 to 90 degrees Celsius, grinding it into a 160-mesh concentrated fine powder, and then mixing the powder into a plaster matrix at a ratio of two to sixty;
Step 3: secondary processing of the raw materials, comprising boiling a sesame oil in the boiling furnace to the extent that dropped water will become a pearl, adding minium at 330 degrees Celsius to make the plaster matrix, removing fire toxin using a process with a combination of water immersion, alcohol and cold storage methods, taking 30 parts by weight of the matrix and putting it into the stove with slow fire to be heated and melted, keeping the temperature between 71° C. and 80° C. to avoid the production of new fire toxin, stirring evenly with one part by weight of the extracted concentrated fine powder or fine medicine, exhausting the moisture and removing from the fire;
Step 4: packaging the finished product by putting the finished product into a packaging bag for packaging, and then putting it into the storage box.
